(19) United States
(12) Patent Application Publication
(10) Pub. No.: US 2011/0111033 A1
(43) Pub. Date: May 12, 2011

(54) HYDROGEL WITH COVALENTLY CROSSLINKED CORE

(76) Inventors: Harald Stover, Dundas (CA); Nicholas Burke, Dundas (CA); M.A. Jafar Mazumder, Hamilton (CA); Feng Shen, Dundas (CA); Murray Potter, Ancaster (CA)

(21) Appl. No.: 12/937,110

(22) PCT Filed: Apr. 9, 2009
(86) PCT No.: PCT/CA09/00448

§ 371 Date: Jan. 20, 2011

Related U.S. Application Data
(60) Provisional application No. 61/071,029, filed on Apr. 9, 2008.

(51) Int. Cl. A61K 35/12; A61K 9/00; A61P 25/00; A61P 3/00; A61P 3/10; A61P 35/00

(52) U.S. Cl. 424/487; 424/484; 424/486; 424/93.7; 424/488

(57) ABSTRACT

A novel hydrogel system is provided. The hydrogel system comprises a biocompatible hydrogel core having dispersed therein a covalently crosslinked polymer matrix. The hydrogel system is useful per se or as an encapsulation system.

### FIELD OF THE INVENTION

The present invention generally relates to the field of hydrogels, and in particular, relates to a hydrogel system that incorporates a stabilizing polyelectrolyte matrix.

### BACKGROUND OF THE INVENTION

Transplantation of encapsulated allogenic and xenogenic cells is a promising approach for the treatment of diseases including, but not limited to, neurological disorders, dwarfism, hemophilia, lysosomal storage disorders, diabetes and cancer. To avoid rejection by the host, the transplanted cells are often protected by a semi-permeable membrane, which allows the exchange of oxygen, nutrients and metabolites, while obscuring the encapsulated cells from the host's immune system.

The most common cell encapsulation system involves the alginate-poly-L-lysine-alginate (APA) microcapsules. These capsules are primarily composed of alginate, a naturally produced polysaccharide composed of β-D-mannuronic acid (M) and α-L-guluronic acid (G) residues. Calcium ions are used to cross-link G-rich regions of the alginate chains. The resulting calcium alginate (CaAlg) hydrogel beads are coated with poly-L-lysine (PLL) to strengthen the outer bead surface and control permeability, followed by coating with a layer of alginate, in order to hide the inflammatory PLL from the host and make the final capsules biocompatible. Barium ions may be used instead of calcium ions, in cases where the neurotoxicity of barium is not an issue. While APA capsules meet many of the requirements for immuno-isolation of cells when implanted into mice, they have shown insufficient strength when implanted into larger animals such as dogs. This may be due to weakening of the hydrogel core by exchange of calcium with other physiological ions and/or the loss of the protective polyelectrolyte coatings.

A number of studies have attempted to address the challenge of long-term mechanical stability by varying the molecular weight or G/M ratio of the alginate, the cross-linking ion, and/or the polyelectrolytes used to coat the capsule. Covalent cross-linking of the coating layer or the alginate core has also been investigated. Another approach has been to examine the use of alternate hydrogel cores, including those made of composite materials. A number of alginate composite materials have been explored for controlled release applications as well as for cell encapsulation. The component(s) blended with alginate may be thermally, ionically or covalently gelled, or may be used to modify viscosity or water content, act as wall-forming materials, control permeability or provide an improved environment for cell growth. For example, capsules suitable for longer-term cell implantation have been made with alginate-cellulose sulfate composite cores where the cellulose sulfate acts as a viscosity modifier and is thought to be a better “wall builder” than alginate when forming polyelectrolyte complexes with the polycations used to coat the capsules. Other approaches use photochemical crosslinking of modified alginate or other macromolecules to form covalently reinforced shells or beads.

However, despite these developments, there remains a need for an improved hydrogel system that overcomes at least one of the disadvantages of currently employed hydrogels for use in, among other things, encapsulation.

### SUMMARY OF THE INVENTION

It has now been discovered that incorporation of reactive polyanion into a biocompatible hydrogel core, followed by exposure of the resulting hydrogel composite to a polycation, yields a hydrogel system comprising a crosslinked polymer network that is readily formed, and advantageously more stable than prior hydrogel systems.

Thus, in one aspect of the invention, a novel hydrogel system is provided comprising a hydrogel that comprises a covalently crosslinked polymer network.

In another aspect, a method of making a hydrogel system is provided. The method comprises the steps of:

i) combining a reactive polyanion with a hydrogel precursor in solution;

ii) contacting the hydrogel solution with a cross-linking agent to form a gel; and

iii) exposing the gel to an aqueous solution comprising a polycation that is reactive with the polyanion to form a hydrogel system comprising a covalently crosslinked polyelectrolyte matrix.

These and other aspects of the invention are described by reference to the detailed description that follows and the drawings.

### BRIEF DESCRIPTION OF THE DRAWINGS

FIG. 1 is a schematic illustrating the different types of crosslinked networks that may be achieved in a hydrogel system according to an aspect of the invention;

FIG. 2 illustrates a representative hydrogel (A), polycation (B) and reactive polyanions (C) useful to prepare a hydrogel system in accordance with an embodiment of the invention;

FIG. 3 graphically illustrates the percentage of reactive polyanion remaining in composite microcapsules at different stages of the capsule preparation;

FIG. 4 illustrates the different abilities of PLL of different molecular weights to diffuse into CaAlg beads;

FIG. 5 is a CLSM image of a core-crosslinked composite capsule (A/A70)PLLr(4-15k, 0.5%)A(0.03%);

FIG. 6 illustrates intensity line profiles from CLSM images of composite microcapsules (0.5% PLL, 4-15 kDa) following in-diffusion of dextran-FITC of MW: a) 10 kDa, b) 70 kDa, c) 150 kDa, d) 250 kDa, and e) 500 kDa.; and

FIG. 7 graphically compares the in vitro cell number per capsule of control APA capsules to shell cross-linked capsules (A/A70)P(0.05% 15-30 kDa)(a) and core cross-linked capsules (A/A70)P(0.5% 4-15 kDa)(b).

### DETAILED DESCRIPTION OF THE INVENTION

A novel hydrogel system is provided comprising a biocompatible hydrogel that comprises a covalently crosslinked polymer matrix.

The term “biocompatible hydrogel” refers to a gel that is compatible with living cells, for example, including cells within a host (e.g. mammal), as well as cells to be transplanted into a host. Suitable such hydrogels generally include water soluble polymers capable of being gelled using biocompatible means such as divalent cation binding and thermal gellation, for example calcium alginate, barium alginate, and hydrogel systems such as those described in Prokop et al. (Adv Polym Sci 1998, 136, 1-51), the contents of which are incorporated herein by reference, for example alginate-cellulose sulphate hydrogel mixtures.

The term “covalently crosslinked” refers to the formation of a covalent bond between reactive polymers that is stable in the presence of an ionic solution (e.g. a sodium chloride solution at a concentration of about 1-2 M), or that is stable at high pH levels, e.g. pH 12-13, such as in the presence of 0.1 N sodium hydroxide. This is in contrast to electrostatic interactions which are commonly labile in the presence of ionic solutions, and at high pH.

The term “polymer matrix” refers to a network of crosslinked biocompatible polymers in the hydrogel, either within the hydrogel core, externally on the hydrogel shell, or both. Suitable polymers to form this matrix include reactive polyelectrolytes, including polyanions containing reactive electrophilic groups, and polycations such as primary and secondary polyamines. As one of skill in the art will appreciate, reactive uncharged polymers may also be used.

The present hydrogel system may be prepared by combining a biocompatible polyanion in solution, for example a physiologically acceptable salt solution such as sodium chloride, with a hydrogel in precursor form in a physiologically acceptable solution, to form a hydrogel-polyanion solution, e.g. a sodium alginate-polyanion solution.

Alternatively, to prepare the present hydrogel system as an encapsulation system, target particles to be encapsulated, e.g. particles to be encapsulated including, but not limited to cells, enzymes, nanoparticles, tissue samples, bacteria, and other entities or life forms that are larger than the pore size of the resulting polymer matrix, are dispersed in a physiologically acceptable salt solution containing the selected hydrogel, in its precursor form. A biocompatible polyanion in a physiologically acceptable solution is then added to the particle-hydrogel solution to form a particle-containing hydrogel-polyanion solution.

To form a cell encapsulation system, live cells are dispersed in a physiologically acceptable salt solution such as 0.9% sodium chloride containing the hydrogel, in its precursor form. A biocompatible polyanion in a similar solution is added to the cell-containing hydrogel to form a cell-hydrogel-polyanion solution, e.g. a cell-sodium alginate-polyanion solution. As one of skill in the art will appreciate, such solutions are prepared under conditions suitable for live cells, including using sterile procedures and materials, at temperatures of about 4° C., and using laminar fumehoods.

Suitable polyanions for use to make the present hydrogel system include polymers, preferably having a molecular weight in the range of about 10 to 500 kDa, and more preferably in the range of 20 to 200 kDa, such as electrophilic polymers in which the electrophilic reactivity is provided by glycidyl methacrylate, aldehyde-containing comonomers, activated esters, acetyl acetonate groups, and other electrophilic monomers such as those having activated double bonds such as acrylate groups and methacrylate groups. These polymers may also contain neutral hydrophilic monomers such as hydroxyethyl methacrylate, hydroxyethyl acrylate, hydroxypropylmethacrylamide, and poly(ethylene glycol) methacrylate; as well as anionic monomers such as acrylic acid and methacrylic acid. In one embodiment, a suitable polyanion is a copolymer of poly(methacrylic acid, sodium salt-co-2-[methacryloyloxy]ethyl acetoacetate) (p(MAA-co-MOEAA)), containing the two constituent monomers in varying ratios, e.g. 70:30, 60:40 and 50:50.

Examples of suitable hydrogels include, as indicated, water soluble polymers capable of being gelled using biocompatible means such as divalent cation binding and thermal gellation, for example calcium alginate and barium alginate. Other high viscosity gel-forming polymers such as cellulose sulphate may be used instead of alginate, or together with alginate, to form the primary hydrogel core.

The amount of polyanion appropriate for inclusion in the system is an amount that does not substantially affect the properties of the hydrogel while being an amount that results in sufficient covalent crosslinking within the core, for example, an amount in the range of about 10 to 200% by weight of the hydrogel, and preferably 20 to 75% by weight of the hydrogel. If polyanions of sufficiently high molecular weight, for example a molecular weight of at least about 250 kDa, or sufficiently high viscosity are used (for example, a viscosity of at least about 30 cps, and preferably higher), these polyanions may themselves serve as the hydrogel precursor as well as the polyanion.

The particle-hydrogel-polyanion solution is then formed into a gel on admixture with an appropriate amount of an ionic gelling agent such as calcium chloride or barium chloride using techniques well-established in the art.

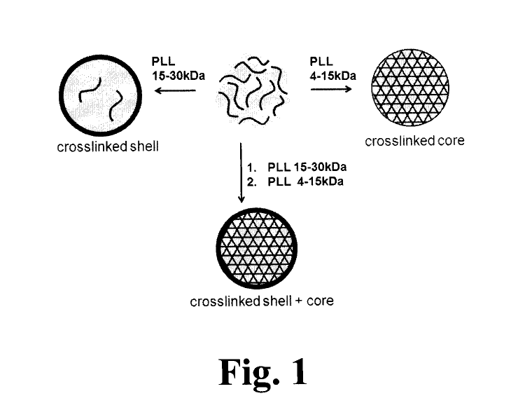

The resulting gel, for example in the form of beads or capsules, is then exposed to a biocompatible polycation that is reactive with the polyanion incorporated within the gel, to result in the desired cross-linking as shown schematically in FIG. 1. Polymer cross-linking within the hydrogel may occur externally on the outer shell of the hydrogel, e.g. the outer layer or surface of the hydrogel which may generally be about 1-100 micrometer in thickness, e.g. 1-50 micrometer in thickness, as well as within the hydrogel core, the internal portion of the hydrogel, e.g. internal to the outer 100 micrometer shell of the hydrogel.

Suitable polycations include those having a molecular weight that balances reactivity with the polyanion in the hydrogel to result in cross-linking, with the capacity to diffuse through the pores of the hydrogel matrix. Thus, as one of skill in the art will appreciate, the appropriate molecular weight of the polycation will depend on the nature of the hydrogel, including composition, concentration and pore size of the hydrogel. Accordingly, suitable polycations include those having a molecular weight that permit their diffusion into the hydrogel core, for example, having a molecular weight in the range of about 1-200 kDa, preferably 2-100 kDa, such as 4-80 kDa, 4-50 kDa, and 4-30 kDa, and most preferably 4-15 kDa, including homopolymers and copolymers based on monomers having primary amine groups such as aminoethyl methacrylate, aminopropylmethacrylamide, aminoethyl acrylate and related monomers. It may be advantageous to use copolymers of amine-containing monomers with 25 to 75 mol % of uncharged hydrophilic comonomers such as hydroxyethyl methacrylate or hydroxypropylmethacrylamide, in order to reduce the positive charge density and thereby prevent inflammatory response on implantation of the system into a host. Other suitable polyamines include polymers such as poly-L-lysine, chitosan, polyornithine and polyethyleneimine.

The amount of polycation appropriate for inclusion in the system is an amount that does not affect the mechanical properties of the hydrogel core while being an amount that results in sufficient covalent crosslinking with the polyanion within the core, for example, an amount that results in at least about a 1:1 stoichiometric functional group ratio between polycation and reactive polyanion. In certain circumstances (e.g. when there is a sufficient amount of both polyanion and polycation to form an extended cross-linked network), as one of skill in the art will appreciate, it may be possible to increase the amount of polyanion without also increasing the amount of polycation such that the ratio of polyanion to polycation may be 2:1 or even 3:1. In order to reduce the tendency of the polycation to bind to the hydrogel, gelling agent such as calcium chloride may optionally be added to the polycation solution at a concentration in the range of about 0.1 to 1.1 wt %, and preferably at a concentration of about 0.3-0.5 wt %.

In one embodiment of the invention, polycations having a first molecular weight, e.g. a molecular weight that is close to or exceeds the molecular weight at which the polycation could diffuse into the hydrogel core (e.g. a molecular weight that prevents, or at least partially prevents, diffusion of the polycation into the hydrogel core), may be exposed to the hydrogel, as well as polycations of a second molecular weight, e.g. a molecular weight that readily permits diffusion of the polycation into the hydrogel. In this embodiment, polycations of the first molecular weight form a protective crosslinked outer shell on the hydrogel system, while the polycations of the second molecular weight diffuse into the hydrogel core to form an internal crosslinked matrix. In this case, the hydrogel is exposed to polycations of the first molecular weight followed by exposure to polycations of the second molecular weight. For a calcium alginate hydrogel, polycations having a molecular weight of at least about 15-30 kDa, or greater, may generally form a crosslinked outer shell on the hydrogel, while polycations having a molecular weight in the range of about 4-15 kDa may generally diffuse into the hydrogel core to result in core crosslinking, forming an internal crosslinked matrix.

Following crosslinking, it may be desirable to enhance the biocompatibility of the crosslinked hydrogel. This may be achieved by treatment, or coating, of the hydrogel to result in a biocompatible polyanionic surface by exposure of the crosslinked hydrogel to a hydrogel precursor solution, for example, sodium alginate 0.05-0.1%, or a reactive polyanion such as those previously identified. Such treatment is particularly desirable when polycations with low biocompatibility are incorporated in the crosslinked hydrogel, e.g. polylysine, as opposed to polycations such as chitosan and amine copolymers which are biocompatible. Following this treatment to enhance biocompatibility, residual electrophilic groups, e.g. of a reactive polyanion coating, may be capped by exposure to biocompatible monoamines or oligoamines, such as amino polyethyleneglycol, glucosamine, or ethanolamine, e.g. a 0.1% solution.

The present method results in a hydrogel system comprising a hydrogel that comprises a covalently cross-linked polymer matrix. The cross-linked polymer matrix functions to stabilize the system, rendering it resistant to both chemical and mechanical challenges, thereby resulting in a hydrogel system having extended implant life in a host. In particular, it is noted that the present crosslinked hydrogel system is more stable to mechanical challenge than uncrosslinked hydrogels as measured by the ability to withstand a compressive force of greater than 50 mNewtons, for example, forces of at least about 100 mNewtons and greater, including compressive forces of at least about 200 mNewtons, preferably compressive forces of at least about 300 mNewtons, more preferably compressive forces of at least about 500 mNewtons, and even more preferably, compressive forcers of about 1000 mNewtons or greater.

The present hydrogel system has widespread utility. At the outset, the cross-linked hydrogel system per se provides a stable, biocompatible, semi-permeable membrane. Among other utilities for such membranes, that would be well-known to those of skill in the art, a crosslinked hydrogel membrane in accordance with the invention is useful in biomolecular separation techniques such as ion exchange and size exclusion chromatography. In this regard, it is noted that this system is not limited to the formation of beads and/or capsules, but may also be prepared as sheets of hydrogel by spin coating or deposition on a flat surface using a spreading knife, gelling using calcium chloride and crosslinking by exposure to the reactive polyamine. In this way, sheets consisting of covalently crosslinked polymer, with or without a target particle, may be prepared.

The present hydrogel system is also useful as a biocompatible coating on devices for implant, including, for example, stents, catheters, other medical implants and the like. In this regard, the device to be coated may be dipped into a polyanion-hydrogel solution, followed by application of a crosslinking polycation.

In addition, the present hydrogel system is useful as an encapsulation system for use in the transplant of cells for the treatment of disease and other conditions requiring cell transplant. The present hydrogel system is also useful to immobilize cells in other environments, for example, in cell culture, and may be used to entrap entities other than cells. In this regard, it is noted that the present hydrogel system may be customized in order to provide a covalently crosslinked polymer network to retain the target entity, e.g. customized to have an average pore size that exceeds the size of the target.

Embodiments of the invention are described in the following specific example which is not to be construed as limiting.

### Example 1

### Materials

Sodium Alginate (Keltone LV, Mₙ=428 kDa) was a gift from the Nutrasweet Kelco Company (San Diego, Calif., USA). Methacrylic acid (MAA, 99%), 2-[methacryloyloxy]ethyl acetoacetate (MOEAA, 95%), poly(methacrylic acid, sodium salt) (PMAANa, Mₙ=5400 Da, 30 wt % solution in water), poly-L-lysine hydrobromide (PLL, Mₙ=15-30 kDa, 4-15 kDa and 1-4 kDa), fluorescein isothiocyanate (FITC)-conjugated bovine serum albumin (Mₙ=66 kDa), fluorescein isothiocyanate-conjugated dextran (Mₙ=10, 70, 150, 250 and 500 kDa), fluorescein isothiocyanate (FITC, 90%), Rhodamine β isothiocyanate (mixed isomers), 2-(N-cyclohexylamino)ethanesulfonic acid (CHES) and trypan blue stain (0.4% in 0.85% saline) were purchased from Sigma-Aldrich, Oakville, ON, and were used as received. 2,2′-Azobis(isobutyronitrile) (AIBN) was purchased from Dupont (Mississauga, ON) and used as received. Sodium chloride (reagent), sodium nitrate (reagent), tetrahydrofuran (THF, reagent) and anhydrous ethyl ether were obtained from Caledon Laboratories Ltd (Caledon, ON). Calcium chloride (Fisher), trisodium citrate dihydrate (Analar, EMD Chemicals, Gibbstown, N.J.) and sodium dihydrogen orthophosphate (BDH, ON) were used as received. Ethanol from Commercial Alcohols (Brampton, ON) and serum free media (SFM) from Gibco (Mississauga, ON) were used as received. Sodium hydroxide and hydrochloric acid solutions were prepared from concentrates (Anachemia Chemical, Rouses Point, N.Y.) by diluting to 0.100 M or 1.000 M with deionized water. The preparation of poly(methacrylic acid-co-2-[methacryloyloxy]ethyl acetoacetate), A70, and its labelling with fluorescein isothiocyanate (FITC) was described previously (Mazumder, M. A. J; Shen, F.; Burke, N. A. D.; Potter, M. A; Stöver, H. D. H, Biomacromolecules, 2008, 9, 2292-2300), the relevant contents of which are incorporated herein by reference. The preparation of poly([2-(methacryloyloxy)ethyl]trimethylammonium chloride), C100 with a molecular weight (M_{w}) of 300 kDa is described in Burke, et al. (Burke, N. A. D.; Mazumder, M. A. J; Hanna, M.; Stöver, H. D. H. J. Polym. Sci. A: Polym. Chem., 2007, 45, 4129-4143), the relevant contents of which are incorporated herein by reference.

Synthesis of PMAANa (MW=40 kDa):

In a typical free radical polymerization, MAA (5.00 g; 58 mmol) and AIBN (95 mg; 0.58 mmol) were dissolved in ethanol (45 mL) in a 60 mL HDPE bottle. The solution was bubbled with nitrogen for several minutes and the bottle was sealed. The mixture was heated in an oven at 60° C. for 24 h while the bottle was rotated at 4 rpm to provide mixing. The polymer was isolated by precipitation in ethyl ether (500 mL), washed with ethyl ether and then dried to constant weight at 50° C. in a vacuum oven. Yield: 4.81 g (96%). PMAANa solutions were prepared by neutralizing PMAA with a stoichiometric amount of 1M NaOH and then diluting to the desired polymer concentration with water.

Poly(methacrylic acid, sodium salt-co-2-[methacryloyloxy]ethyl acetoacetate) (p(MAA-co-MOEAA); 90:10 (A90), 80:20 (A80), 60:40 (A60) and 50.50 (A50):

Poly(methacrylic acid, sodium salt-co-2-[methacryloyloxy]ethyl acetoacetate) copolymers were prepared by free radical polymerization as previously described for A70 (Mazumder et al., 2008). For example, MAA (7.84 g, 91.02 mmol), MOEAA (2.28 g, 10.11 mmol), and AIBN (166 mg, 1.01 mmol, 1 mol %) were heated at 60° C. in ethanol (100 ml) for 24 h, followed by precipitation in diethyl ether, to give 9.38 g (94%) P(MAA-co-MOEAA), A90. A80 (ethanol, yield: 85%), A60 (THF, yield: 71%) and A50 (1:1 THF/ethanol, yield: 85%) were prepared in a similar fashion.

Poly(methacrylic acid, sodium salt-co-2-[methacryloyloxy]ethyl acetoacetate) (p(MAA-co-MOEAA), 70:30 (A70) of different molecular weights.

Poly(methacrylic acid, sodium salt-co-2-[methacryloyloxy]ethyl acetoacetate), p(MAA-co-MOEAA) 70:30 were prepared as described previously (Mazumder et al. 2008). Monomer to initiator ratios of 20:1, 100:1 and 800:1 were used, and resulted in A70 of 22, 42 and 149 kDa, respectively.

### Rhodamine-Labelled Poly-L-lysine (PLLr):

Poly-L-lysine (1-4k, 4-15k, or 15-30k, 55.5 mg, 0.265 mmol of lysine units) was dissolved in 5 ml 0.1M NaHCO3 buffer solution at pH 9 in a 20 ml glass vial. Rhodamine isothiocyanate (2.7 mg, 0.005 mmol) dissolved in 0.5 ml DMF was added to the PLL solution and the mixture was stirred for 1 hour at 20° C. The resulting solution was dialysed against deionized water using a cellulose acetate membrane (Spectrum Laboratories, 3.5 kDa MW cut-off for 4-15k and 15-30k PLL and 1 kDa MW cut-off for 1-4 kDa PLL) for one week. The dialysed polymer solution was freeze-dried, and the polymer dried further to constant weight in a vacuum oven at 50° C. Final label contents were determined by UV/Vis spectroscopy, and found to be 0.76, 0.77 and 0.62 mol % of the total monomer units of 1-4k, 4-15k and 15-30k PLL, respectively. Final yields of isolated, labelled polymer were 10, 56 and 40% for the 1-4k, 4-15k and 15-30k PLL, respectively.

### Molecular Weight Determination:

Molecular weights of the PMAANa (A100-40k), p(MAA-co-MOEAA), and Dextran-FITC samples were determined by gel permeation chromatography (GPC) with a system consisting of a Waters 515 HPLC pump, Waters 717 plus Autosampler, three Ultrahydrogel columns (0-3 kDa, 0-50 kDa, 2-300 kDa), and a Waters 2414 refractive index detector. Samples were eluted with a flow rate of 0.8 mL/min and the system was calibrated with commercially available narrow dispersed molecular weight polyethylene glycol (PEG) standards (Waters, Mississauga, ON).

Dextran-FITC samples were eluted with 0.1M NaNO₃, while for A50-A100, the mobile phase was 0.3 M NaNO₃ in 0.05 M phosphate buffer (pH 7). All anionic polymer solutions for GPC analysis were prepared by the addition of stoichiometric amounts of 1 M NaOH to the MAA-containing precursor polymer followed by dilution with the mobile phase. Preparation of Ca (alginate-A70) Composite Beads:

The Ca-(Alginate-A70) composite microbeads were prepared following the procedure described by Ross et al. (Hum. Gene Ther. 2002, 11, 2117-2127), the contents of which are incorporated herein by reference. Sodium alginate (0.045 g), and A70 or A70f (0.015 g) were dissolved in 3.0 g saline solution (0.9% NaCl) to form a solution containing 1.5 wt % Na alginate and 0.5 wt % A70 or A70f. The pH was adjusted to 7 with 0.1M NaOH. The solutions were filtered with sterile filters (0.45 μm, Acrodisc Syringe Filter, Pall Corporation, USA). A syringe pump (Orion sage pump, model #M362) was used to extrude this solution through a 27-gauge blunt needle (Popper & Sons, New York) at a rate of 99.9 mL/hr. A concentric airflow (4 L/min) passing by the needle tip is used to induce droplet formation. The droplets were collected in 30 mL of 1.1 wt % calcium chloride/0.45% sodium chloride gelling bath. The resulting Ca-(alginate-A70) composite beads were washed in sequence with four-fold volumes of a) 1.1% CaCl₂, 0.45% NaCl for 2 minutes; b) 0.55% CaCl₂, 0.68% NaCl for 2 minutes; c) 0.28% CaCl₂, 0.78% NaCl for 2 minutes; d) 0.1% CHES, 1.1% CaCl₂, 0.45% NaCl for 3 minutes; and then e) 0.9% NaCl for 2 minutes and stored in saline.

Coating of Ca(alginate-A70) Composite Beads with Poly-L-Lysine and Sodium Alginate:

A dense suspension of Ca (alginate-A70) composite beads (3 mL) was exposed to 10 ml of 0.05% (w/v) poly-L-lysine (PLL, pH=8) for 6 minutes and washed once each with x ml of a) 0.1% CHES, 1.1% CaCl₂, 0.45% NaCl for 3 minutes, b) 1.1% CaCl₂, 0.45% NaCl for 2 minutes and c) 0.9% saline for 2 minutes. The resulting Ca (Alginate-A70)-PLL beads were then coated with 10 ml of 0.03% (w/v) sodium alginate for 4 minutes, followed by three washes with 10 mL of 0.9% saline. The final composite capsules were stored in the last saline solution.

### Capsule Characterization:

Capsules and polyelectrolyte complexes were examined with an Olympus BX51 optical microscope fitted with a Q-Imaging Retiga EXi digital camera and ImagePro software. The average diameters of the beads and capsules were determined by analyzing three batches of approximately 50-100 beads or capsules each.

Phase contrast microscope images were taken using a Wild M40 microscope, and confocal images were taken with a confocal laser scanning microscope (CLSM) consisting of air-cooled Argon and HeNe lasers (LASOS; LGK 7628-1), ZEISS microscope (LSM 510) and LSM Image browser software (version 3.5).

### Chemical and Mechanical Stress Test:

Dense microcapsule suspensions in saline (100 μl) were placed in 15 ml polypropylene conical tubes and exposed to 5% w/v (170 mM) sodium citrate for 5 minutes, followed by exposure to 3M sodium chloride. The tubes were attached to a wheel placed at an angle of 30 degrees from horizontal, and rotated at 30 rpm for 15 minutes at room temperature. The beads were then washed with water and treated with trypan blue to stain the polycations. Morphological change was observed by optical microscopy.

### Cell Culture:

The cell line used was the C₂C₁₂ cell line (American Type Culture Collection [ATCC], Rockville, Md.; Catalogue No. CRL-1772). The cells were maintained in Dulbecco's Modified Eagle's Medium (DMEM) supplemented with 10% fetal bovine serum (Gibco, Grand Island, N.Y.) and 100 U/mL penicillin-100 μg/mL streptomycin (Gibco, Grand Island, N.Y.) in the presence of 5% CO₂ with 100% humidity at 37° C. in a water jacket incubator.

### Cell Viability:

The number of viable cells per capsule was determined with an Alamar Blue assay as described in Li, A. A, McDonald, et al. Sci. Polym. Ed, 2003, 14, 533-549, the relevant portion of which is incorporated herein by reference. 100 μL of capsules were loaded in a 24-well plate with 500 μL media. 50 μL of alamar blue was added to each sample and the plate was incubated at 37° C. for 4 hours. After incubation, 100 μL of supernatant was taken from each well and placed in a microtiter plate. The fluorescence of each sample was read with a Cytofluor II fluorimeter, with an excitation wavelength of 530 nm and an emission wavelength of 590 nm. The number of viable cells was determined by comparing the fluorescence intensity with a standard curve generated from a known number of cells. The test was performed in triplicate.

### Permeability Measurements:

Capsule permeability was evaluated using fluorescein-labelled dextran (dextran-FITC) or bovine serum albumin (BSA-FITC). The procedure with dextran-FITC was a modified version of the procedure described in Vandenbossche, G. M. R, et al. (Pharmacol. 1991, 43, 275-277), the contents of which are incorporated by reference, and employed samples having nominal MWs of 10, 70, 150, 250 and 500 kDa. In the case of dextran-FITC, the capsules (0.2 g) were suspended in saline (0.2 mL), exposed to 1.0 mL of 0.0015% dextran-FITC for 24 h at room temperature. When BSA-FITC was employed, the capsules (0.5 g) were suspended in saline, exposed to 5 mL of 0.05% BSA-FITC for 24 h at room temperature, and then washed 5 times with saline to remove free protein.

The microcapsules were then examined by confocal laser scanning microscopy (CLSM). 100 μL of microcapsule suspension was placed on a microscope slide within a Teflon ring (7 mm dia., 3 mm depth) and images were obtained at the capsule equator. Intensity profiles were obtained from the CLSM images with a 25-pixel wide line using UTHSCSA Image Tool software (version 3.0).

### Results:

The ability of polyelectrolytes to form crosslinked networks throughout an alginate bead, leading to formation of a permanent three-dimensional support structure for cell encapsulation is described.

This involved adding the reactive polyanion, A70, to the sodium alginate solution, prior to introduction onto the calcium chloride gelling bath. The resulting primary calcium alginate beads thus contain A70 homogeneously distributed throughout. Subsequent exposure of these beads to aqueous solutions of poly-L-lysine (PLL) led to the formation of crosslinked A70-PLL networks in different horizons within the beads, depending on the molecular weights and mobilities of the polyelectrolytes used. For example, use of 15-30 kDa PLL led to formation of thin (5-30 micron) outer shells comprised of crosslinked polyelectrolytes. Use of lower molecular weight PLL (such as 4-15 kDa) at an appropriate concentration, led to diffusion of the PLL throughout the hydrogel core and efficient crosslinking of the reactive polyanion present in the core.

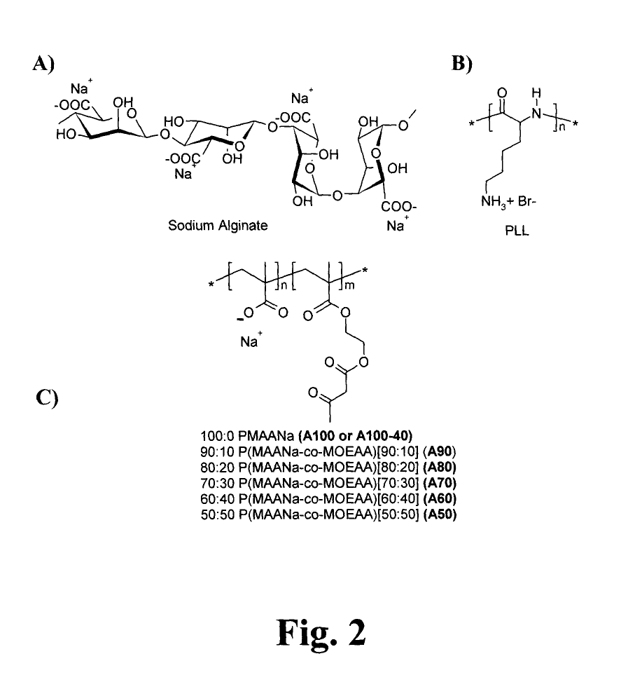

The structures of the polyelectrolytes are shown in FIG. 2 and the properties of the synthetic polyelectrolytes are described in Table 1. Monomer to initiator ratios of 20:1, 100:1 and 800:1 were used to obtain A70 having molecular weights of 22, 42 and 149 kDa, respectively. Attempts to prepare higher MW polymer resulted in gellation, attributed to covalent crosslinking during polymerization.

TABLE 1
Polymer properties

| Polymer | Mₙ (kg/mol)^{a} | MAA:MOEAA^{b} |
|---|---|---|
| A100 | 5.4 | — |
| A100f | 29 | — |
| A100-40k | 40 | — |
| A90 | 29 | 89:11 |
| A80 | 41 | 79:21 |
| A70-22k | 22 | 70:30(±3) |
| A70-42k | 42 | 70:30(±3) |
| A70-149k | 149 | 70:30(±5) |
| A60 | 32 | 64:36 |
| A50 | 31 | 44:56 |

^{a}Mₙ determined by Gel Permeation Chromatography.
^{b}mol % composition determined by ¹H NMR using a Bruker AV 200 spectrometer for samples dissolved in DMSO.

Fluorescently-labelled versions of A70, PLL and A100 were prepared by reaction with FITC (A70f) or rhodamine β isothiocyanate (PLLr) or via copolymerization with fluorescein O-methacrylate (A100f). The resulting A70f-22k, A70f-42k and A70f-149k were found to have 0.22, 0.34 and 0.32 mol %, respectively, of their total monomer units labelled with fluorescein. Unless specifically noted, A70 or A70f with a molecular weight of 42 kDa were employed.

The initial step in all such capsule formations is gelling of the alginate/polyanion mixture in CaCl₂. CaAlg is a solid gel that can resist moderate mechanical stresses. In contrast, the calcium complexes of the synthetic polyanion A 100, prepared by similarly combining 1 wt % solutions of the polyanions with excess 100 mM CaCl₂, resulted only in formation of liquid coacervate droplets. The higher molecular weight A100-40k, A90 and A80 gave similar but more viscous liquid complexes. When A70 (22, 42 or 149 kDa), A60 or A50 was mixed with CaCl₂ no macroscopic phase separation was observed, likely due to the lower carboxylate content of the polymer.

The absence of solid gels formed from these polyanions reflects their weaker calcium binding as compared to the cooperative “egg box” calcium complexation characteristic of alginates, and suggests these polyanions should retain some mobility even within calcium alginate beads.

### Ca-[Alginate (1.5%)-A70 (0.5%)] Composite Beads:

Calcium alginate beads containing the synthetic polyanions were prepared by dripping mixtures of sodium alginate and the polyanion into a CaCl₂ bath. The composite beads formed from a solution containing 1.5 wt % sodium alginate (Keltone LV) and 0.5 wt % A70 or its fluorescein-labelled analogue (A70f) had an average diameter of 650 μm and appeared identical to those formed from sodium alginate alone.

The Ca(Alg/A70f) composite beads were exposed to 0.05% poly-L-lysine (PLL), washed with saline and then coated with a 0.03% sodium alginate solution to obtain final capsules with an anionic, biocompatible surface. When examined by optical microscopy the capsules looked similar to the uncoated beads but the surface was easily stained by trypan blue indicating the presence of the polycation. In addition, the surface appeared pink when a Rhodamine-labelled PLL was used to coat the composite beads.

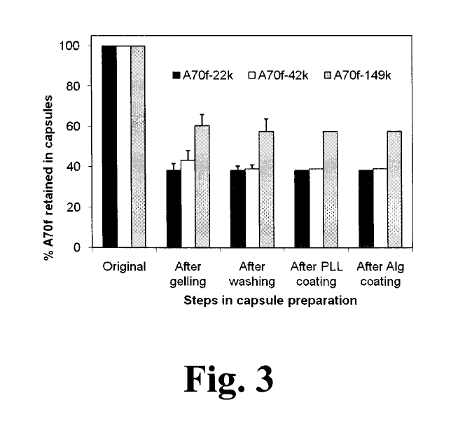

Following capsule preparation, the CaCl₂ gelling bath and the solutions used during coating/washing were analyzed by UV-visible spectroscopy for the presence of A70f. It was found that ˜60±5% of the original A70f-22k or A70f-42k was lost during gelling and the initial set of washes (see FIG. 3). In contrast, only ˜40±5% of the higher MW A70f-149k was lost during gelling and the initial set of washes. No additional A70f loss was observed during the subsequent PLL coating process. Uncoated capsules stored in a roughly 6-fold excess of saline at 4° C. had lost an additional 3% of the original A70f after 2 days, and 16% after 3 months (not shown). PLL/alginate-coated capsules did not lose a significant amount of A70f over 8 months storage.

Thus, a significant amount of A70 is lost principally in the gellation step during which the droplets shrink to about 60% of their original volume. Core liquid is expelled from the gelling beads along with any polymer chains that are not physically entangled or ionically cross-linked within the CaAlg gel. Use of higher MW A70 increases the percent retention of the polymer. The preferred molecular weight of the polyanion is between about 10 to 500 kDa, and more preferably 40-200 kDa. Higher molecular weights are desirable as they help provide the viscosity needed to maintain the droplet shape during gellation in the calcium chloride gelling bath. It should be recognized that losses of A70 and analogous polymers from the hydrogels may be larger when hydrogels with larger pore sizes are used.

CLSM showed that A70f is initially homogeneously distributed within the Ca(A/A70f) beads. Images obtained 1-2 hours after coating these beads with PLL and sodium alginate, show in addition a very thin outer shell formed by concentration of some of the A70f in the form of a PLL/A70f crosslinked shell.

When the coated capsules were treated with excess 170 mM (5% w/v) sodium citrate to liquefy the CaAlg core, the capsules swelled (40-50% diameter increase), indicating the absence of significant core crosslinking. Model studies suggested that formation of a crosslinked A70/PLL network requires equimolar or greater amounts of PLL relative to A70. Accordingly, fluorescently labelled PLL was used to track the in-diffusion of the polycation and determine the fraction that reaches the core of the beads.

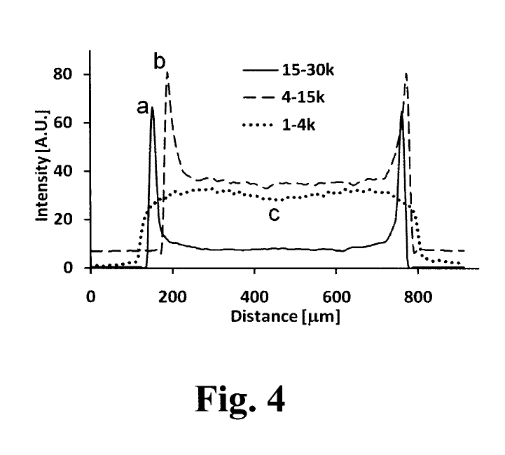

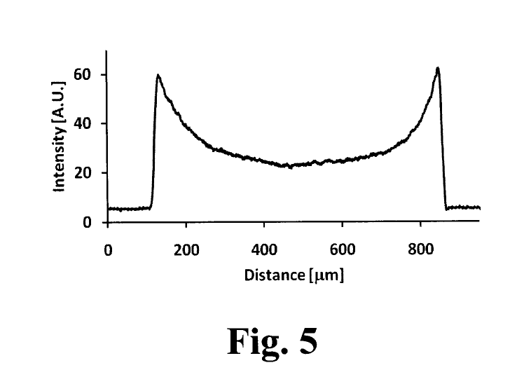

CLSM images of capsules coated with 0.05% solutions of PLLr show that higher MW PLLr (15-30 kDa) is concentrated near the capsule surface, while the lowest MW PLLr (1-4k) is evenly distributed throughout the composite microcapsules (FIG. 5). The intermediate MW PLLr (4-15 kDa) showed both formation of a distinct shell, and significant in-diffusion to the core of the bead (FIG. 4). The shell formed by 4-15 kDa PLLr was thicker (36 μm width at half-height of the line-out shown in FIG. 4) than that formed by the 15-30 kDa PLLr, (23 μm), which is consistent with the deeper penetration expected of this intermediate MW polycation. Hence, reacting Ca(A/A70) beads with PLL of appropriate MW and concentrations, leads to capsules that are reinforced both internally and externally.

The integrity of uncoated and PLL-coated Ca-(Alg/A70) composite beads in the presence of sodium citrate and sodium chloride was examined by optical microscopy (OM) and compared with that of classical APA microcapsules. Uncoated beads composed of Ca-(Alg/A70) or Ca-Alg dissolve when exposed to 170 mM (5% w/v) sodium citrate, a good calcium chelator. In contrast, addition of sodium citrate to PLL(15-30k)-coated capsules such as APA or Ca(A/A70)PA caused the core of the beads to dissolve, while the shells consisting of the polyelectrolyte complex survived. One test for covalent crosslinking of the shell is to expose citrate-treated capsules to 2 M NaCl while vigorously agitating, which dissolves (in the case of 1-4 and 4-15k PLL) or noticeably weakens (in the case of 15-30k PLL) shells held together by just ionic interactions, compared to their crosslinked analogs. Another test, especially for the higher MW PLL, involves exposing the shells to 0.1 M sodium hydroxide, which neutralizes the ammonium ions of PLL leading to rapid and almost complete dissociation of all electrostatic PECs used here.

This showed that APA has an ionically cross-linked shell, which dissolves at high ionic strength as well as at high pH. On the other hand, the shells surrounding the Ca-(Alg/A70) PA composite microcapsules were covalently cross-linked, and survived the challenge with high ionic strength and high pH.

After exposure of a composite bead coated with 0.05% PLL (4-15 kDa). to sodium citrate followed by 2 M sodium chloride, the capsule was manually cut, revealing both a thin crosslinked shell and A70f diffusing out through the hole in the shell. The shell was self-supporting, but it was clear that the core of the bead was not crosslinked, likely due to the presence of insufficient amounts of PLL.

Composite beads coated with low MW PLL (1-4 kDa, 0.05%) which had PLLr homogeneously distributed throughout the beads, dissolved within seconds upon exposure to sodium citrate (70 mM). This indicates that although this low MW PLL readily penetrates the interior of the beads, at the present concentration of 0.05% it is unable to crosslink the A70 to the extent necessary to give a crosslinked shell or core. The chains may be too short to effectively bridge between A70 chains. A sodium citrate concentration of 70 mM was found to be sufficient to extract calcium from both CaAlg and composite beads, and was used henceforth.

### Core-Crosslinked (A/A70)PA Capsules

For Ca(A/A70(42 kDa)) beads that retain roughly 40% of their original A70 loading, exposure to 0.05 w % PLL corresponds to a ratio of crosslinking groups (amine/acetoacetate) of about 2:1. UV/Vis analysis of a supernatant PLL (15-30 kDa) solution after coating showed that only half of this PLL was actually absorbed by the capsules and, thus, the PLL-coated beads have an overall amine/acetoacetate ratio of approximately 1:1. However, much of this bound PLL was involved in electrostatic complexation and is concentrated in the dense shell at the surface as shown in FIG. 4. This indicates that the effective amine/acetoacetate ratio in the core is much lower, and explains the absence of core-crosslinking in the resulting capsules.

Analysis of the in-diffusion patterns (FIG. 4) indicated that the intermediate MW PLL (4-15 kDa) represented a good compromise between ease of in-diffusion and a MW high enough to crosslink the A70 in the core, provided it is available in sufficiently high concentration to compensate for incomplete capture and preferential binding to the shell.

Accordingly, the PLL (4-15k) concentration was increased from 0.05% to 0.5 and 1%. Coating using 1% PLL solution resulted in wrinkling of the bead surface, while 0.5% PLL (4-15 kDa) resulted in smooth bead surfaces. (A/A70f)PA capsules coated with 0.5% PLL (4-15k), followed by alginate (0.03%) were manually cut, and exposed to 70 mM citrate and then 2 M sodium chloride. The capsules undergo little swelling and there is minimal loss of A70f demonstrating that sufficient PLL has diffused into the core to crosslink the bead throughout. The crosslinked beads also survived subsequent treatment with 0.1M NaOH.

The above-identified experiment was repeated with capsules formed using A100f, instead of A70f, in order to exclude the possibility that electrostatic bonding could hold the core together during these challenges. The resulting (A/A100f)P(4-15k, 0.5%)A(0.03%) capsules swelled considerably when exposed to 70 mM citrate for about 5 min. The outer layer, consisting of an electrostatic complex of PLL with alginate and A100f, appeared to swell more than the inner core as revealed when the shell is cut. Subsequent exposure to 2 M sodium chloride completely dissolved both shell and core within three minutes confirming again that the permanent structure for the A70-containing capsule is indeed based on covalent crosslinking.

The location of the PLL is interesting to note as it is important for both crosslinking and biocompatibility. Accordingly, Ca(A/A70) beads were coated with 0.5% PLLr (4-15 kDa) and then examined by fluorescence microscopy and CLSM (FIG. 5). Capsules exposed to citrate and manually crushed, followed by the addition of 2 M NaCl underwent only some swelling and showed minor loss of PLLr, confirming the role of PLL in the covalent crosslinking of both shell and core. The presence of a distinct PLLr shell in addition to core crosslinking indicates that the higher MW fraction of PLLr(4-15 kDa) is limited to forming a surface network, while the lower MW fraction can diffuse into the core to crosslink with A70. The presence of a distinct shell after exposure to 2 M NaCl, indicates that it does not involve electrostatic binding of excess PLL to alginate, but rather covalent bonding, to A70.

CLSM analysis of a (A/A70)PLLr (4-15k, 0.5%)A (0.03%) capsule shows that the concentration of PLL fell more gradually moving from shell to core (FIG. 5) as compared to the analogous beads coated with 0.05% PLLr (4-15 kDa), the intensity profile of which is shown in FIG. 4. The width at half height was close to 100 micrometer.

The exposure of the Ca(A/A70) beads to PLL(0.5%) in 1.1% CaCl₂, 0.45% NaCl instead of the standard 0.9% saline, did not lead to significantly increased in-diffusion of PLL (images not shown).

The capsule shell plays important roles in permeation and biocompatibility and, thus, it may be advantageous to carry out shell formation independent of the core-crosslinking process. In order to test the scope for separately controlling core-crosslinking and shell formation, CaAlg beads were sequentially coated with two PLL solutions of different MWs. Ca(A/A70f) beads were first exposed to 0.05% PLL (15-30k) for 1 min, followed without washing by another exposure to 0.5% PLL (4-15k) for 6 min, and after a wash step, by the usual final coat with 0.03% Alg for 4 min. The resulting capsules, after manual cutting and exposure to citrate and 2 M NaCl, showed both the presence of an outer shell formed by reaction of the higher MW PLL with A70f near the surface, and core-crosslinking between the lower MW PLL and A70f in the core. In contrast, the capsules prepared using only 0.5% PLL (4-15k) did not show a distinct outer shell. This demonstrates the ability of the two-stage approach to give some independent control over shell and core crosslinking, and may enable tuning of the MW cut-off as required for specific cell immuno-isolations.

The final outer coating of sodium alginate may be advantageously replaced with a final outer coating of 0.05% A70. The resulting covalent attachment of the outer polyanion should provide better long-term protection against recognition of the polycation by the host. This outer polyanionic coat may consist of A70 analogs incorporating PEG side chains, such as may be introduced into A70 by copolymerization with PEG methacrylate. Alternatively, the final capsules may be treated for a short period of time with a dilute solution of PEG amines, or amino sugars such as glucosamine in order to cap residual acetoacetate groups and reduce the likelihood of adverse protein binding to the outer coating during incubation of post-transplant.

### MW Cut-Off of APA and Composite Microcapsules:

The MW cut-off of these new shell- and core-crosslinked capsules, e.g. the molecular weight of components that can readily diffuse into the capsules as opposed to the molecular weight of components that cannot diffuse into the capsules, was estimated using a series of commercial dextran-FITC samples with nominal molecular weights of 10, 70, 150, 250 and 500 kDa. Gel permeation chromatography (GPC) analysis showed that the samples had broad MW distributions, with polydispersity indices of 4.7 for the 150 kDa sample and approximately 2 for the other samples (Table 2). Note that the MWs measured in this GPC analysis are lower than the nominal MWs likely because the linear poly(ethylene glycol) standards used for calibration have different hydrodynamic radii than dextrans of similar MW. The broad MW distributions mean that in-diffusion of the low MW fraction may occur with each of the samples. In addition, dextran may behave differently than globular proteins in solution, and as such the use of dextran-FITC provides only a rough indication of the MW cut-off.

TABLE 2
Molecular weight and polydispersity
index of Dextran-FITC samples.

| Nominal MW | Apparent | Apparent | Apparent | |
|---|---|---|---|---|
| (kDa) | Mₙ (kDa)^{a} | M_{w} (kDa)^{a} | Mₚ (kDa)^{a} | PDI |
| 10 | 4.8 | 9.4 | 10.6 | 1.94 |
| 70 | 29 | 56 | 52.8 | 1.93 |
| 150 | 14 | 66.1 | 40.2 | 4.69 |
| 250 | 66.8 | 144.4 | 160.5 | 2.16 |
| 500 | 87.2 | 192.9 | 211 | 2.21 |

^{a}MW as determined with a PEG calibration curve.

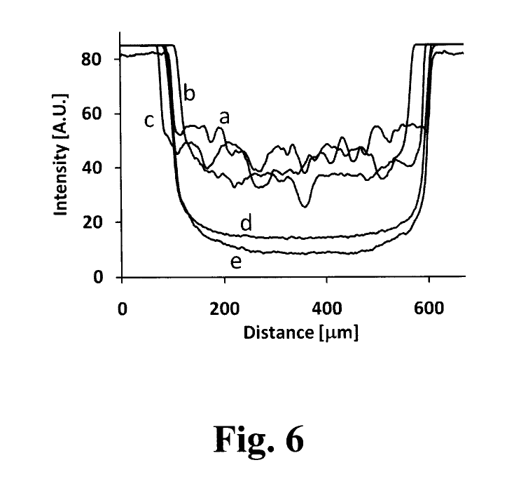

Shell-crosslinked (A/A70)P(15-30k, 0.05%)A capsules exposed to 0.0015% dextran-FITC solutions for 24 h and then examined by CLSM showed increasing in-diffusion with decreasing MW (data not shown). Similarly, core-crosslinked (A/A70)P(4-15k, 0.5%)A capsules containing C₂C₁₂ mouse myoblast cells exposed to dextran-FITC (0.05%) for 24 h (FIG. 6) showed that dextrans of 500 and 250 kDa are almost completely excluded, while dextrans having MW's of 10 and 70 kDa can diffuse in freely. At least some fraction of the 150 kDa dextran also diffused into the capsules. Both the shell- and core-crosslinked capsules have MW cut-offs that are roughly 100 to 200 kDa, similar to APA capsules (data not shown), and in line with data reported for other capsules.

The permeability of APA and shell-crosslinked (A/A70)P(15-30k)A microcapsules containing cells was also assessed by looking for the uptake of BSA-FITC (MW 66 kDa). Both types of microcapsules were permeable to BSA-FITC, indicating a MW cut-off greater than 70 kDa, consistent with the dextran-FITC results.

### In Vitro Cell Viability:

C₂C₁₂ mouse cells were encapsulated in APA capsules, the shell-crosslinked (A/A70)P(15-30k, 0.05%)A capsules and the core-crosslinked (A/A70)P(4-15k, 0.5%)A capsules. The capsules were cultured in vitro for one week and the numbers of living cells per capsule were determined with the Alamar Blue assay. Note that the cell viability tests on the two new types of capsules were performed at different times each with an APA control, to take into account variables affecting cell growth that are unrelated to the presence of the new materials. The average live cell numbers in shell-crosslinked capsules are similar to those in APA capsules over the week long incubation, indicating that the A70 in the core of the (A/A70)PA capsules is not detrimental to cell viability.

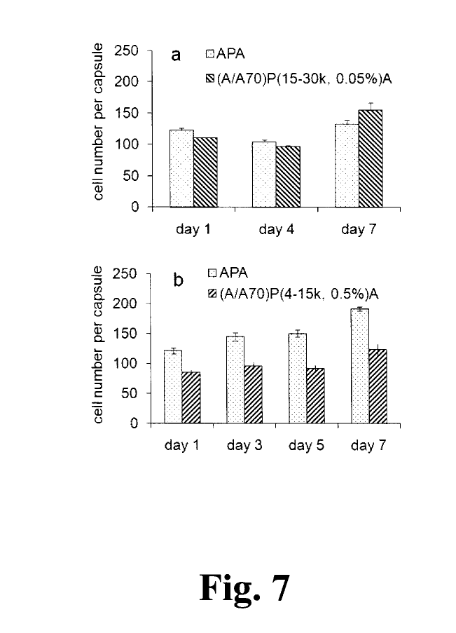

The cell viability results for the core-crosslinked (A/A70)PA capsule are shown in FIG. 7. The APA capsules show higher cell numbers throughout the incubation although similar relative increases in cell numbers (50-60%) are seen for the two types of capsule. Comparison with the higher cell numbers observed in the case of analogous shell-crosslinked capsules prepared using only 0.05% PLL(15-30 kDa) suggests that the lower initial cell viability in the present capsules is due to the larger amount of lower MW PLL used.

The diffusion of PLL into the capsule core did not have a detrimental effect on cell viability. The location of PLLr (4-15 kDa) in the shell, compared to the homogeneous distribution of A70f (FIG. 4) reflects the fact that PLL is applied from the outside, while A70 is found throughout the core. PLL in the bead core may exhibit reduced toxicity towards the encapsulated cells because most of the PLL diffusing in from the outside reacts with the A70 present throughout the core and residual unreacted PLL may complex with alginate thereby reducing any cytotoxic effect.

A PLL:A70 ratio of about 1:1 or higher is desirable in order to form crosslinked complexes.

### Example 2

### Effect of Polyanion Loading on Crush Force

Five types of composite capsules were prepared from saline containing 1.5 wt % sodium alginate together with either 0.5 or 1.0% A70, crosslinked by exposure to 0.5% PLL (4-15k), followed by a final exposure to either 0.1% sodium alginate or 0.1% A70 (Table 3). The beads, and enough of the solution to cover them, were then placed in a custom-built texture analyzer consisting of a 4 square millimeter silicon wafer attached to a piezo-electric transducer. The wafer was positioned over one bead at a time and moved down vertically at a constant speed of 10 μm/s with the help of a stepper motor while plotting the force registered against vertical displacement.

Compression data were corrected both for the buoyancy of the silicon wafer, and the elastic give of the experimental setup. Upon compression, the beads deformed to between 2 and 2.5 times their original diameter, and about 20 to 25% of their original height, before cracking. It was noted that the present core-crosslinked beads, upon exceeding their maximum compressive loading, do not undergo catastrophic failure, but rather undergo progressive cracking that still provides some matrix isolation for the cells embedded in the fragments. This is in contrast to many APA type core-shell capsules that fail by a catastrophic bursting mechanism, which exposes all of the bead content to the host.

The net loading force at which each of the beads showed failure by cracking is shown in Table 3. A bead prepared from saline containing 1.5 wt % sodium alginate together with either 0.5 A70, crosslinked by exposure to 0.5% PLL (4-15k), followed by a final exposure to 0.1% sodium alginate exhibited a first crack at about 120 mNewton of compressive force (entry 1 in table 3). Compression of the same type of bead after extraction of the calcium in the core with sodium citrate shows a slight reduction in load at failure, indicating that most of the bead strength derives from the synthetic polymer network, rather than from the calcium alginate matrix (entry 2). Extraction with citrate is designed to mimic the slow exchange of calcium for sodium known to take place in tissue. The results indicate that the beads strength of these covalently crosslinked capsules should not suffer from such ion exchange.

Using 0.1% A70 instead of 0.1% alginate for the outer coating further increases the load at failure to 350 mNewton (entry 3), indicating that additional covalent crosslinking takes place between the PLL and the outer layer of A70. Increasing the core loading of A70 to 1% from 0.5% also increases the load at failure to above 1000 mNewton before citrate treatment (entry 4), and to 450 mNewton after citrate treatment (entry 5). Typical force at failure for uncrosslinked APA capsules is between 20 and 40 mNewtons, with the failure mechanism resembling the sudden bursting of a balloon, rather than progressive cracking.

TABLE 3

| | [Alg] | [A70] | | | Citrate- | Max force | |
|---|---|---|---|---|---|---|---|
| | in core | in core | PLL | Outer | treated | at failure | Failure |
| # | (wt %) | (wt %) | (4-15 kDa) | Coating | [170 mM] | [mNewton] | mode |
| 1 | 1.5 | 0.5 | 0.5 | 0.1% | No | 120 ± 20 | Cracking |
| | | | | Alg | | | |
| 2 | 1.5 | 0.5 | 0.5 | 0.1% | Yes | 105 | Cracking |
| | | | | Alg | | | |
| 3 | 1.5 | 0.5 | 0.5 | 0.1% | No | 350 (± 50) | Cracking |
| | | | | A70 | | | |
| 4 | 1.5 | 1.0 | 0.5 | 0.1% | No | >1000 | — |
| | | | | Alg | | | |
| 5 | 1.5 | 1.0 | 0.5 | 0.1% | Yes | 450 | Cracking |
| | | | | Alg | | | |

1. A hydrogel system comprising a hydrogel core that comprises a covalently crosslinked polymer matrix.

2. A hydrogel system as defined in claim 1, wherein the crosslinked polymer matrix comprises a polyanion-polycation network.

3. A hydrogel system as defined in claim 2, wherein the polyanion in the matrix has a molecular weight in the range of about 10 to 500 kDa.

4. (canceled)

5. A hydrogel system as defined in claim 3, wherein the polyanion comprises an electrophilic polymer comprising an electrophilic entity selected from the group consisting of a glycidyl methacrylate, an aldehyde-containing comonomer, an activated ester, an acetyl acetonate group and an activated double bond.

6. A hydrogel system as defined in claim 3, wherein the polyanion comprises one or more monomers selected from the group consisting of hydroxyethyl methacrylate, hydroxyethyl acrylate, hydroxypropylmethacrylamide, poly(ethylene glycol) methacrylate; acrylic acid, and methacrylic acid.

7. A hydrogel system as defined in claim 3, wherein the polyanion is a copolymer of poly(methacrylic acid, sodium salt-co-2-[methacryloyloxy]ethyl acetoacetate) (p(MAA-co-MOEAA)).

8. A hydrogel system as defined in claim 2, wherein the polycation in the matrix has a molecular weight in the range of about 1-200 kDa.

9. (canceled)

10. A hydrogel system as defined in claim 8, wherein the polycation is selected from the group consisting of a polymer comprising an amine-containing monomer, optionally combined with an uncharged hydrophilic comonomer, poly-L-lysine, chitosan, polyethyleneimine and polyornithine.

11. A hydrogel system as defined in claim 1, wherein the hydrogel core is selected from the group consisting of an alginate, cellulose sulphate and an alginate-cellulose sulphate mixture.

12. (canceled)

13. (canceled)

14. A hydrogel system as defined in claim 1, wherein the crosslinked polymer matrix is formed in at least one of the hydrogel core and the hydrogel shell.

15. A hydrogel system as defined in claim 1, which is able to withstand a compressive force of at least about 100 mNewtons.

16. A method of making a hydrogel system comprising the steps of:
i) combining a reactive polyanion with a hydrogel precursor in solution;
ii) contacting the hydrogel solution with a cross-linking agent to form a gel; and
iii) exposing the gel to an aqueous solution comprising a polycation that is reactive with the polyanion in an amount sufficient to form a hydrogel system comprising a covalently crosslinked polymer matrix.

17. A method as defined in claim 16, wherein the polyanion is combined with the hydrogel precursor in amount in the range of about 10 to 200% by weight of the hydrogel.

18. A method as defined in claim 16, wherein the cross-linking agent is selected from the group consisting of calcium chloride or barium chloride.

19. A method as defined in claim 16, wherein the polycation is added in an amount that results in about a 1:1 stoichiometric functional group ratio between polycation and reactive polyanion.

20. A method as defined in claim 16, wherein the gel is exposed to a first polycation having a first molecular weight that does not permit the diffusion of the first polycation into the hydrogel, followed by exposure of the gel to a second polycation having a second molecular weight that permits diffusion of the second polycation into the hydrogel.

21. (canceled)

22. A method as defined in claim 16, wherein the hydrogel precursor solution comprises a target particle.

23. A method as defined in claim 20, wherein the target particle is selected from the group consisting of cells, enzymes, nanoparticles, bacteria, and particles larger than the pore size of the hydrogel system.

24. A hydrogel as defined in claim 1, wherein the hydrogel encapsulates a target particle.

25. A hydrogel as defined in claim 22, wherein the target particle is selected from the group consisting of cells, enzymes, nanoparticles, bacteria, and particles larger than the pore size of the hydrogel system.

26. (canceled)
